(19) United States
(12) Patent Application Publication
(10) Pub. No.: US 2014/0290659 A1
(43) Pub. Date: Oct. 2, 2014

(54) REVERSING VALVE AND HIGH FREQUENCY OSCILLATION AIRFLOW GENERATOR

(71) Applicant: KONINKLIJKE PHILIPS N.V., EINDHOVEN (NL)

(72) Inventors: Tongqing Chen, Shanghai (CN); Wei Zhou, Shanghai (CN); Huimin Chen, Shanghai (CN)

(21) Appl. No.: 14/362,927

(22) PCT Filed: Dec. 7, 2012
(86) PCT No.: PCT/IB2012/057056

§ 371 Date: Jun. 5, 2014

(30) Foreign Application Priority Data
Dec. 14, 2011 (CN) PCTCN2011083972

(51) Int. Cl. A61M 16/20; F16K 11/07

(52) U.S. Cl. CPC A61M 16/20; F16K 11/0716
USPC 128/205.24; 137/625.3

(57) ABSTRACT

The invention provides a reversing valve for reversing a fluid flow, comprising a valve housing defining a cylindrical chamber, at least four passage outlets being formed in the valve housing and opening into the cylindrical chamber, and further comprising a valve core being received rotatably and hermetically within the cylindrical chamber of the valve housing, at least two passages being defined in the valve core, wherein the passage outlets and the passages are arranged such that the reversing valve can reverse the liquid flow once every time that the valve core rotates through 90°. The invention also provides a high frequency oscillation airflow generator. According to the invention, the reversing frequency of the gas flow and thus the efficiency of the high frequency oscillation airflow generator are improved.

### FIELD OF THE INVENTION

The present invention relates to providing high frequency variable pressure to a patient to treat a respiratory disorder, and, in particular, to a reversing valve and a high frequency oscillation airflow generator comprising such a reversing valve.

### BACKGROUND OF THE INVENTION

A ventilation technique commonly known as “high frequency ventilation” is widely used to augment a patient's respiratory rate to assist with the patient's breathing and/or to remove an accumulated secretion from the patient's respiratory system. According to this technique, the pressure of the gas flow delivered to the patient's respiratory system oscillates between two levels at a relatively high frequency. Hence, high oscillation frequency is critical for this technique.

U.S. Pat. No. 6,708,690B1 discloses an apparatus for providing high frequency variable pressure to a patient. The apparatus comprises a gas circuit, a valve disposed in the gas circuit, a driving assembly for driving the valve and two blowers disposed in the gas circuit. The valve includes a generally cylindrical valve member having a first axial surface, a second axial surface and a side surface. A first passage that extends from the first axial surface to a first portion of the side surface and a second passage that extends from the second axial surface to a second portion of the side surface are defined in the cylindrical member. When the valve is rotated to be in a first position and a second position that are spaced 180° apart around the central axis of the cylindrical valve member, the apparatus generates respectively a positive pressure that delivers a gas flow to the patient's respiratory system and a negative pressure that delivers a gas flow from the patient's respiratory system. Every time that the driving assembly for driving the valve rotates through 360°, the valve reverses the gas flow twice.

To improve the efficiency of providing a high frequency variable pressure to the patient, there is a need to design a novel reversing valve to meet the requirement that the reversing frequency of the gas flow is as high as possible.

### SUMMARY OF THE INVENTION

Accordingly, it is an object of the present invention to provide a reversing valve and a high frequency oscillation airflow generator comprising such a reversing valve that overcomes the shortcomings of conventional pressure oscillation techniques.

According to one aspect of the present invention, there is provided a reversing valve for reversing a fluid flow comprising:
a valve housing defining a cylindrical chamber, at least four passage outlets being formed in the valve housing and opening into the cylindrical chamber; and
a valve core being received rotatably and hermetically within the cylindrical chamber of the valve housing, at least two passages being defined in the valve core;
wherein the passage outlets and the passages are arranged such that the reversing valve can reverse the liquid flow once every time that the valve core rotates through 90°.

Preferably, four passage outlets opening into the cylindrical chamber are formed in the valve housing, a first passage outlet and a second passage outlet thereof are diametrically opposite to each other, a third passage outlet and a fourth passage outlet thereof are diametrically opposite to each other, a line connecting the third passage outlet and the fourth passage outlet is parallel to a line connecting the first passage outlet and the second outlet. The valve core is a cylindrical valve core, in which two passages are defined, wherein a first passage and a second passage of the two passages are spaced apart and extend perpendicularly to each other, and both the first passage and the second passage extend through and are perpendicular to a central axis of the cylindrical valve core, and the distance between the line connecting the third passage outlet and the fourth passage outlet and the line connecting the first passage outlet and the second outlet is equal to that between the first passage and the second passage.

Preferably, eight passage outlets opening into the cylindrical chamber are formed in the valve housing, a first passage outlet and a second passage outlet thereof are diametrically opposite to each other, a third passage outlet and a fourth passage outlet thereof are diametrically opposite to each other and a second line connecting the third passage outlet and the fourth passage outlet is parallel to a first line connecting the first passage outlet and the second passage outlet, a fifth passage outlet and a sixth passage outlet are diametrically opposite to each other and a third line connecting the fifth passage outlet and the sixth passage outlet is parallel to the first line, a seventh passage outlet and an eighth passage outlet are diametrically opposite to each other and a fourth line connecting the seventh passage outlet and the eighth passage outlet is parallel to the first line. The valve core is a cylindrical and hollow valve core forming a central passage, one end of the central passage is closed and the opposite end is open, a first group of holes including four first holes, a second group of holes including two second holes, a third group of holes including four third holes, and a fourth group of holes including two fourth holes are formed in a wall of the cylindrical and hollow valve core, and each group of holes is spaced from every other group of holes along a central axis of the cylindrical and hollow valve core, the four first holes are distributed equidistantly in a first plane perpendicular to the central axis of the cylindrical and hollow valve core, two diametrically opposite first holes thereof are in communication with each other via a conduit to form a first passage, the two second holes are distributed so as to be diametrically opposite in a second plane perpendicular to the central axis of the cylindrical and hollow valve core, a line connecting with the two second holes is parallel to the first passage, the two second holes are in communication with each other via a conduit to form a second passage, the four third holes are distributed equidistantly in a third plane perpendicular to the central axis of the cylindrical and hollow valve core, a line connecting with two diametrically opposite third holes thereof is perpendicular to the first passage, the two diametrically opposite third holes are in communication with each other via a conduit to form a third passage, the two fourth holes are distributed diametrically opposite in a fourth plane perpendicular to the central axis of the cylindrical and hollow valve core, a line connecting with the two fourth holes is perpendicular to the first passage, the two fourth holes are in communication with each other via a conduit to form a fourth passage, the distances between the first line and the second line, between the second line and the third line and between the third line and the fourth line are equal to those between the first plane and the second plane, between the second plane and the third plane and between the third plane and the fourth plane, respectively.

Preferably, four passage outlets opening into the cylindrical chamber are formed in the valve housing and distributed equidistantly along a circumferential direction. The valve core comprises two circular bodies and a spacer centrally interconnecting said two circular bodies when the valve core is received rotatably and hermetically within the cylindrical chamber of the valve housing to form the reversing valve, and the two circular bodies, the spacer and the valve housing together define a first passage and a second passage.

According to another aspect of the present invention, there is provided a high frequency oscillation airflow generator comprising a reversing valve for reversing a gas flow, at least one blower, a driving assembly for rotatably driving the reversing valve and a plurality of connecting lines connecting the reversing valve with the at least one blower, the reversing valve comprising:
a valve housing defining a cylindrical chamber, at least four passage outlets being formed in the valve housing and opening to the cylindrical chamber; and
a valve core being received rotatably and hermetically within the cylindrical chamber of the valve housing, at least two passages being defined in the valve core;
wherein the passage outlets and the passages are arranged such that the reversing valve can reverse the gas flow once every time that the driving assembly drives the valve core to rotate through 90°.

Preferably, four passage outlets opening into the cylindrical chamber are formed in the valve housing, a first passage outlet and a second passage outlet thereof are diametrically opposite to each other, a third passage outlet and a fourth passage outlet thereof are diametrically opposite to each other, a line connecting the third passage outlet and the fourth passage outlet is parallel to a line connecting the first passage outlet and the second outlet. The valve core is a cylindrical valve core, two passages are defined in the cylindrical valve core, a first passage and a second passage of the two passages are spaced from and perpendicular to each other, both the first passage and the second passage extend through and are perpendicular to a central axis of the cylindrical valve core, and the distance between the line connecting the third passage outlet and the fourth passage outlet and the line connecting the first passage outlet and the second outlet is equal to that between the first passage and the second passage. The at least one blower comprises a first blower and a second blower, the plurality of connecting lines comprises a first connecting line, a second connecting line and a third connecting line, the first connecting line has a free end and connects at the opposite end with the second passage outlet and the fourth outlet, the second connecting line connects at one end with the first passage outlet and at the opposite end with an outlet of the first blower, the third connecting line connects at one end with the third passage outlet and at the opposite end with an inlet of the second blower, and an inlet of the first blower and an outlet of the second blower open into the atmosphere or other gas source.

Preferably, eight passage outlets opening into the cylindrical chamber are formed in the valve housing, a first passage outlet and a second passage outlet thereof are diametrically opposite to each other, a third passage outlet and a fourth passage outlet thereof are diametrically opposite to each other, and a second line connecting the third passage outlet and the fourth passage outlet is parallel to a first line connecting the first passage outlet and the second passage outlet, a fifth passage outlet and a sixth passage outlet are diametrically opposite to each other, and a third line connecting the fifth passage outlet and the sixth passage outlet is parallel to the first line, a seventh passage outlet and an eighth passage outlet are diametrically opposite to each other, and a fourth line connecting the seventh passage outlet and the eighth passage outlet is parallel to the first line. The valve core is a cylindrical and hollow valve core forming a central passage, one end of the central passage is closed and the opposite end is open, a first group of holes including four first holes, a second group of holes including two second holes, a third group of holes including four third holes, and a fourth group of holes including two fourth holes are formed in a wall of the cylindrical and hollow valve core, each group of holes is spaced from every other group of holes along a central axis of the cylindrical and hollow valve core, the four first holes are distributed equidistantly in a first plane perpendicular to the central axis of the cylindrical and hollow valve core, two diametrically opposite first holes thereof are in communication with each other via a conduit to form a first passage, the two second holes are distributed so as to be diametrically opposite in a second plane perpendicular to the central axis of the cylindrical and hollow valve core, a line connecting with the two second holes is parallel to the first passage, the two second holes are in communication with each other via a conduit to form a second passage, the four third holes are distributed equidistantly in a third plane perpendicular to the central axis of the cylindrical and hollow valve core, a line connecting with two diametrically opposite third holes thereof is perpendicular to the first passage, the two diametrically opposite third holes are in communication with each other via a conduit to form a third passage, the two fourth holes are distributed so as to be diametrically opposite in a fourth plane perpendicular to the central axis of the cylindrical and hollow valve core, a line connecting with the two fourth holes is perpendicular to the first passage, the two fourth holes are in communication with each other via a conduit to form a fourth passage, the distances between the first line and the second line, between the second line and the third line and between the third line and the fourth line are equal to those between the first plane and the second plane, between the second plane and the third plane and between the third plane and the fourth plane, respectively. The at least one blower comprises one blower, the plurality of connecting lines comprises a first connecting line, a second connecting line, a third connecting line, a fourth connecting line and a fifth connecting line, the first connecting line has a free end and connects at opposite ends with the third passage outlet and the seventh passage outlet, the second connecting line connects at one end with the fifth passage outlet and at the opposite end with an inlet of the blower, the third connecting line connects at one end with an outlet of the blower and at the opposite end with the first passage outlet, the fourth connecting line connects at one end with the second passage outlet and at the opposite end with the fourth passage outlet, and the fifth connecting line connects at one end with the sixth passage outlet and at the opposite end with the eighth passage outlet, the free end of the central passage of the hollow valve core opens into the atmosphere or other gas source.

Preferably, four passage outlets opening into the cylindrical chamber are formed in the valve housing and distributed equidistantly along a circumferential direction. The valve core comprises two circular bodies and a spacer centrally interconnecting said two circular bodies; when the valve core is received rotatably and hermetically within the cylindrical chamber of the valve housing to form the reversing valve, the two circular bodies, the spacer and the valve housing together define a first passage and a second passage. The at least one blower comprises one blower, the plurality of connecting lines comprises a first connecting line and a second connecting line, the first connecting line connects at one end with an outlet of the blower and at the opposite end with a first passage outlet of the four passage outlets, the second connecting line connects at one end with a second passage outlet of the four passage outlets which is diametrically opposite to the first passage outlet and at the opposite end with an inlet of the blower, one of a third passage outlet and a fourth passage outlet remains unobstructed and the other one opens into the atmosphere or other gas source.

Preferably, a reversing frequency of the gas flow generated by the high frequency oscillation airflow generator is adjusted by changing a rotating frequency of the driving assembly.

Preferably, the flow rate of the gas flow generated by the high frequency oscillation airflow generator is adjusted by changing the rotational speed of the blower.

These and other objects, features and characteristics of the present invention, as well as the methods of operation and functions of the related elements of structure and the combination of parts and economies of manufacture, will become more apparent upon consideration of the following description and the appended claims with reference to the accompanying drawings, all of which form part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a perspective view of a valve core of a reversing valve according to a first embodiment of the present invention;

FIG. 2 is a perspective view of a valve housing of a reversing valve according to a first embodiment of the present invention;

FIG. 3 is a schematic diagram of a high frequency oscillation airflow generator comprising a reversing valve according to a first embodiment of the present invention, in which the high frequency oscillation airflow generator generates a negative pressure to deliver a gas flow from a patient's respiratory system;

FIG. 4 is a schematic diagram of a high frequency oscillation airflow generator comprising a reversing valve according to a first embodiment of the present invention, in which the high frequency oscillation airflow generator generates a positive pressure to deliver a gas flow to a patient's respiratory system;

FIG. 5 is a perspective view of a valve core of a reversing valve according to a second embodiment of the present invention;

FIG. 6 is a perspective view of a valve housing of a reversing valve according to a second embodiment of the present invention;

FIG. 7 is a schematic diagram of a high frequency oscillation airflow generator comprising a reversing valve according to a second embodiment of the present invention, in which the high frequency oscillation airflow generator generates a negative pressure to deliver a gas flow from a patient's respiratory system;

FIG. 8 is a schematic diagram of a high frequency oscillation airflow generator comprising a reversing valve according to a second embodiment of the present invention, in which the high frequency oscillation airflow generator generates a positive pressure to deliver a gas flow to a patient's respiratory system;

FIG. 9 is an exploded perspective view of a reversing valve according to a third embodiment of the present invention;

FIG. 10 is a schematic diagram of a high frequency oscillation airflow generator comprising a reversing valve according to a third embodiment of the present invention, in which the high frequency oscillation airflow generator generates a negative pressure to deliver a gas flow from a patient's respiratory system; and

FIG. 11 is a schematic diagram of a high frequency oscillation airflow generator comprising a reversing valve according to a third embodiment of the present invention, in which the high frequency oscillation airflow generator generates a positive pressure to deliver a gas flow to a patient's respiratory system.

### DETAILED DESCRIPTION OF THE PREFERRED EMBODIMENTS

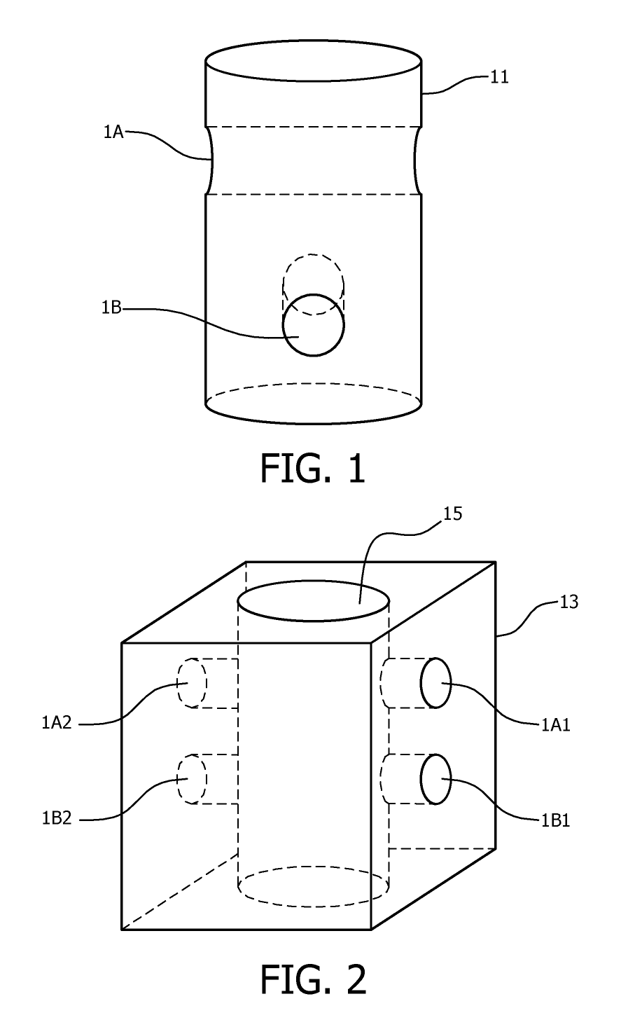

FIG. 1 is a perspective view of a valve core of a reversing valve according to a first embodiment of the present invention and FIG. 2 is a perspective view of a valve housing of a reversing valve according to a first embodiment of the present invention. As shown in FIGS. 1 and 2, a reversing valve 10 according to a first embodiment of the present invention comprises a cylindrical valve core 11. The cylindrical valve core 11 defines a first passage 1A and a second passage 1B. The first passage 1A and the second passage 1B are spaced from and perpendicular to each other. Both the first passage 1A and the second passage 1B extend through and are perpendicular to a central axis of the cylindrical valve core 11.

The reversing valve 10 further comprises a valve housing 13. The valve housing 13 defines a cylindrical chamber 15. Four passage outlets 1A1, 1A2, 1B1 and 1B2 opening into the cylindrical chamber 15 are formed in the valve housing 13. The first passage outlet 1A1 and the second passage outlet 1A2 are diametrically opposite to each other. The third passage outlet 1B1 and the fourth passage outlet 1B2 are diametrically opposite to each other. A line connecting the third passage outlet 1B1 and the fourth passage outlet 1B2 is parallel to a line connecting the first passage outlet 1A1 and the second passage outlet 1A2. The distance between the line connecting the third passage outlet 1B1 and the fourth passage outlet 1B2 and the line connecting the first passage outlet 1A1 and the second passage outlet 1A2 is equal to that between the first passage 1A and the second passage 1B. Thus, the first passage 1A may align with the first passage outlet 1A1 and the second passage outlet 1A2 and the second passage 1B may align with the third passage outlet 1B1 and the fourth passage outlet 1B2 when the cylindrical valve core 11 is received rotatably and hermetically within the cylindrical chamber 15 of the valve housing 13 to form the reversing valve 10.

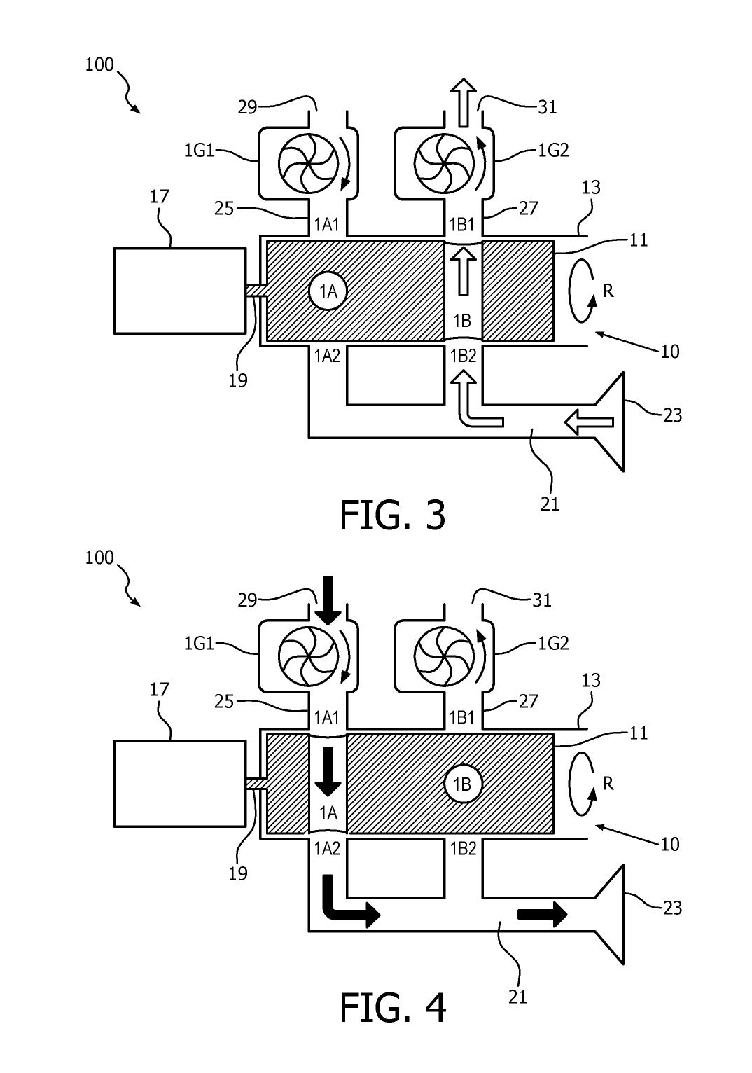

FIG. 3 is a schematic diagram of a high frequency oscillation airflow generator comprising a reversing valve according to a first embodiment of the present invention, in which the high frequency oscillation airflow generator generates a negative pressure to deliver a gas flow from a patient's respiratory system. FIG. 4 is a schematic diagram of a high frequency oscillation airflow generator comprising a reversing valve according to a first embodiment of the present invention, in which the high frequency oscillation airflow generator generates a positive pressure to deliver a gas flow to a patient's respiratory system. As shown in FIGS. 3 and 4, the high frequency oscillation airflow generator 100 comprises the reversing valve 10 according to the first embodiment of the present invention, a first blower 1G1, a second blower 1G2 and a driving assembly 17 for rotatably driving the reversing valve 10. The driving assembly 17 is coupled with the cylindrical valve core 11 via a shaft 19. The driving assembly 17 may be an electrical motor. However, driving assembly 17 may also be a pneumatic or hydraulic motor.

The high frequency oscillation airflow generator 100 also comprises a first connecting line 21 which has a free end 23 and which connects at the opposite end with two of four passage outlets of the reversing valve 10, for example, the second passage outlet 1A2 and the fourth outlet 1B2. The high frequency oscillation airflow generator 100 further comprises a second connecting line 25 which connects at one end with the first passage outlet 1A1 of the reversing valve 10 and at the opposite end with an outlet of the first blower 1G1 and a third connecting line 27 which connects at one end with the third passage outlet 1B1 of the reversing valve 10 and at the opposite end with an inlet of the second blower 1G2. An inlet 29 of the first blower 1G1 and an outlet 31 of the second blower 1G2 open into the atmosphere. Of course, the inlet 29 of the first blower 1G1 and an outlet 31 of the second blower 1G2 may open into any other suitable gas source.

When the high frequency oscillation airflow generator 100 is in operation, the driving assembly 17 drives the cylindrical valve core 11 to rotate hermetically within the valve housing 13 along a direction indicated by arrow R. When the cylindrical valve core 11 rotates to a position as shown in FIG. 3, the first passage 1A of the reversing valve 10 is blocked by the valve housing 13 while the second passage 1B communicates with the third passage outlet 1B1 and the fourth passage outlet 1B2. At this moment, under the action of the second blower 1G2, the gas flow flows along a direction indicated by a hollow arrow from the free end 23 of the first connecting line 21 to the outlet 31 of the second blower 1G2 through the first connecting line 21, the second passage 1B and the third connecting line 27.

When the cylindrical valve core 11 further rotates through 90° from the position as shown in FIG. 3 to a position as shown in FIG. 4, the first passage 1A of the reversing valve 10 communicates with the first passage outlet 1A1 and the second passage outlet 1A2, while the second passage 1B is blocked by the valve housing 13. At this moment, under the action of the first blower 1G1, the gas flow flows along a direction indicated by a solid arrow from the inlet 29 of the first blower 1G1 to the free end 23 of the first connecting line 21 through the second connecting line 25, the first passage 1A and the first connecting line 21. The gas flow is reversed at the free end 23 of the first connecting line 21 compared with that shown in FIG. 3.

When the cylindrical valve core 11 further rotates through 90° from the position as shown in FIG. 4, the reversing valve 10 returns to the position as shown in FIG. 3. This process is repeated periodically. The free end 23 of the first connecting line 21 usually couples to an airway of a patient so that the high frequency oscillation airflow generator 100 generates alternately a negative pressure and a positive pressure to deliver the gas flow from and to the patient's respiratory system, respectively. Obviously, every time that the driving assembly 17 drives the cylindrical valve core 11 to rotate through 360°, the reversing valve 10 reverses the gas flow at the free end 23 of the first connecting line 21 four times. In other words, every time that the driving assembly 17 drives the cylindrical valve core 11 to rotate through 90°, the reversing valve 10 reverses the gas flow once at the free end 23 of the first connecting line 21.

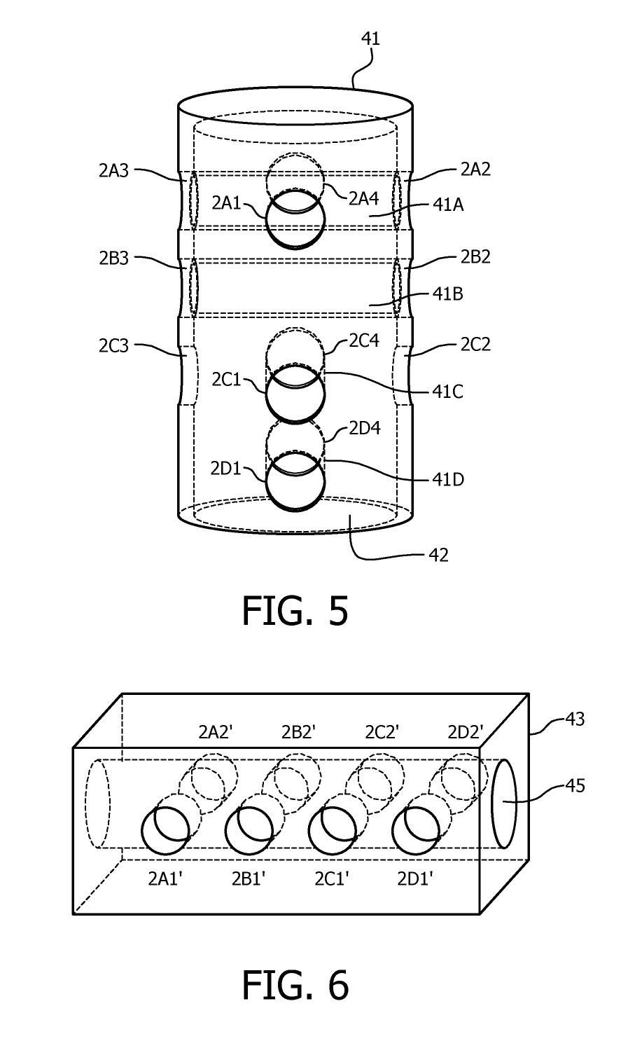

FIG. 5 is a perspective view of a valve core of a reversing valve according to a second embodiment of the present invention and FIG. 6 is a perspective view of a valve housing of a reversing valve according to a second embodiment of the present invention. As shown in FIGS. 5 and 6, a reversing valve 40 according to a second embodiment of the present invention comprises a cylindrical and hollow valve core 41 to form a central passage 42. One end of the central passage 42 is closed and the opposite end is open. A first group of holes including four first holes 2A1, 2A2, 2A3 and 2A4, a second group of holes including two second holes 2B2 and 2B3, a third group of holes including four third holes 2C1, 2C2, 2C3 and 2C4, and a fourth group of holes including two fourth holes 2D1 and 2D4 are formed in the wall of the hollow valve core 41. Each group of holes is spaced from every other group of holes along a central axis of the cylindrical and hollow valve core 41. Four first holes 2A1, 2A2, 2A3 and 2A4 are distributed equidistantly in a first plane perpendicular to the central axis of the cylindrical and hollow valve core 41. Two diametrically opposite first holes 2A2 and 2A3 are in communication with each other via a conduit to form a first passage 41A. Two second holes 2B2 and 2B3 are distributed so as to be diametrically opposite in a second plane perpendicular to the central axis of the cylindrical and hollow valve core 41. A line connecting with two second holes 2B2 and 2B3 is parallel to the first passage 41A. Two second holes 2B2 and 2B3 are in communication with each other via a conduit to form a second passage 41B. Four third holes 2C1, 2C2, 2C3 and 2C4 are distributed equidistantly in a third plane perpendicular to the central axis of the cylindrical and hollow valve core 41. A line connecting with two diametrically opposite third holes 2C1 and 2C4 is perpendicular to the first passage 41A. The two diametrically opposite third holes 2C1 and 2C4 are in communication with each other via a conduit to form a third passage 41C. Two fourth holes 2D1 and 2D4 are distributed so as to be diametrically opposite in a fourth plane perpendicular to the central axis of the cylindrical and hollow valve core 41. A line connecting with two fourth holes 2D1 and 2D4 is perpendicular to the first passage 41A. The two fourth holes 2D1 and 2D4 are in communication with each other via a conduit to form a fourth passage 41D.

The reversing valve 40 further comprises a valve housing 43. The valve housing 43 defines a cylindrical chamber 45. Eight passage outlets 2A1′, 2A2′, 2B1′, 2B2′, 2C1′, 2C2′, 2D1′ and 2D2′ opening into the cylindrical chamber 45 are formed in the valve housing 43. The first passage outlet 2A1′ and the second passage outlet 2A2′ are diametrically opposite to each other. The third passage outlet 2B1′ and the fourth passage outlet 2B2′ are diametrically opposite to each other and a second line connecting the third passage outlet 2B1′ and the fourth passage outlet 2B2′ is parallel to a first line connecting the first passage outlet 2A1′ and the second passage outlet 2A2′. The fifth passage outlet 2C1′ and the sixth passage outlet 2C2′ are diametrically opposite to each other and a third line connecting the fifth passage outlet 2C1′ and the sixth passage outlet 2C2′ is parallel to the first line connecting the first passage outlet 2A1′ and the second passage outlet 2A2′. The seventh passage outlet 2D1′ and the eighth passage outlet 2D2′ are diametrically opposite to each other and a fourth line connecting the seventh passage outlet 2D1′ and the eighth passage outlet 2D2′ is parallel to the first line connecting the first passage outlet 2A1′ and the second passage outlet 2A2′. The distances between the first line and the second line, between the second line and the third line and between the third line and the fourth line are equal to those between the first plane and the second plane, between the second plane and the third plane and between the third plane and the fourth plane, respectively. Thus, when the cylindrical and hollow valve core 41 is received rotatably and hermetically within the cylindrical chamber 45 of the valve housing 43 to form the reversing valve 40, two diametrically opposite holes of four first holes 2A1, 2A2, 2A3 and 2A4 may align with the first passage outlet 2A1′ and the second passage outlet 2A2′ respectively, two second holes 2B2 and 2B3 may align with the third passage outlet 2B1′ and the fourth passage outlet 2B2′ respectively, two diametrically opposite holes of four third holes 2C1, 2C2, 2C3 and 2C4 may align with the fifth passage outlet 2C1′ and the sixth passage outlet 2C2′ respectively, two fourth holes 2D1 and 2D4 may align with the seventh passage outlet 2D1′ and the eighth passage outlet 2D2′ respectively.

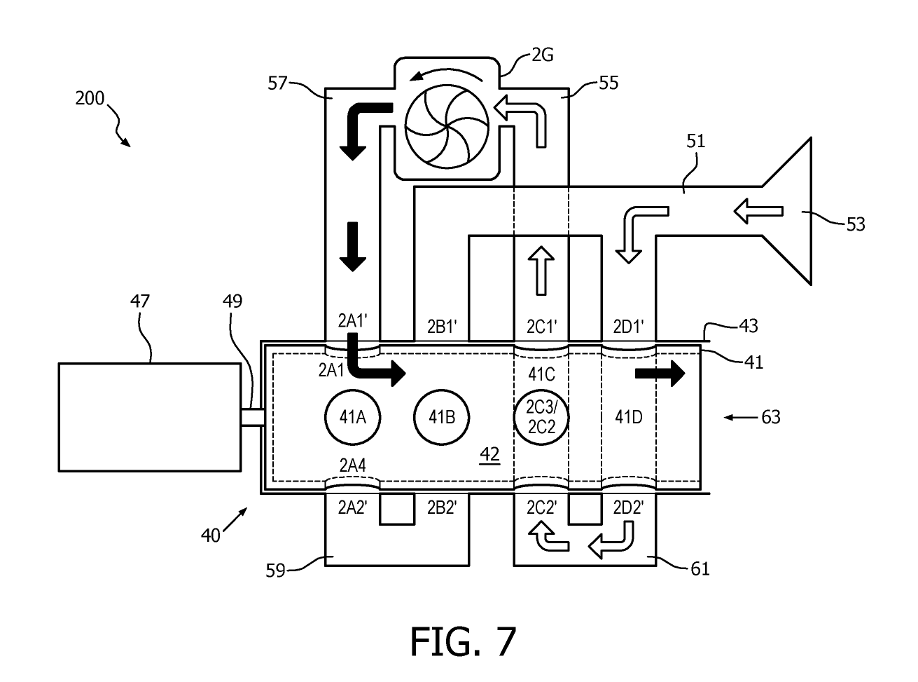

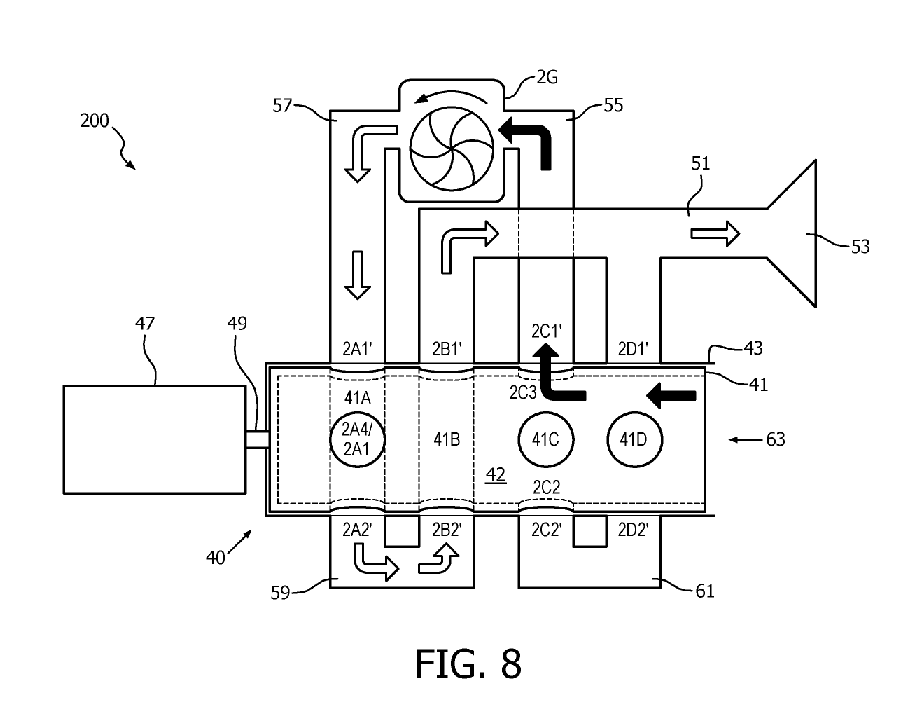

FIG. 7 is a schematic diagram of a high frequency oscillation airflow generator comprising a reversing valve according to a second embodiment of the present invention, in which the high frequency oscillation airflow generator generates a negative pressure to deliver a gas flow from a patient's respiratory system. FIG. 8 is a schematic diagram of a high frequency oscillation airflow generator comprising a reversing valve according to a second embodiment of the present invention, in which the high frequency oscillation airflow generator generates a positive pressure to deliver a gas flow to a patient's respiratory system. As shown in FIGS. 7 and 8, the high frequency oscillation airflow generator 200 comprises the reversing valve 40 according to the second embodiment of the present invention, a blower 2G, and a driving assembly 47 for rotatably driving the reversing valve 40. The driving assembly 47 is coupled with the cylindrical and hollow valve core 41 via a shaft 49. The driving assembly 47 may be an electrical motor. However, driving assembly 47 may also be a pneumatic or hydraulic motor.

The high frequency oscillation airflow generator 200 also comprises a first connecting line 51 which has a free end 53 and which connects at the opposite end with two out of eight passage outlets of the reversing valve 40, for example, the third passage outlet 2B1′ and the seventh passage outlet 2D1′. The high frequency oscillation airflow generator 200 further comprises a second connecting line 55 which connects at one end with the fifth passage outlet 2C1′ of the reversing valve 40 and at the opposite end with an inlet of the blower 2G, a third connecting line 57 which connects at one end with an outlet of the blower 2G and at the opposite end with the first passage outlet 2A1′ of the reversing valve 40, a fourth connecting line 59 which connects at one end with the second passage outlet 2A2′ of the reversing valve 40 and at the opposite end with the fourth passage outlet 2B2′ of the reversing valve 40, and a fifth connecting line 61 which connects at one end with the sixth passage outlet 2C2′ of the reversing valve 40 and at the opposite end with the eighth passage outlet 2D2′ of the reversing valve 40. The free end 63 of the central passage 42 of the hollow valve core 41 opens to the atmosphere. Of course, the free end 63 of the central passage 42 may open into any other suitable gas source.

When the high frequency oscillation airflow generator 200 is in operation, the driving assembly 47 drives the cylindrical and hollow valve core 41 to rotate hermetically within the valve housing 43. When the cylindrical and hollow valve core 41 rotates to a position as shown in FIG. 7, the first connecting line 51 communicates with the fifth connecting line 61 via the fourth passage 41D between two fourth holes 2D1 and 2D4, the fifth connecting line 61 communicates with the second connecting line 55 via the third passage 41C between two diametrically opposite third holes 2C1 and 2C4, the third connecting line 57 communicates with the free end 63 of the central passage 42 via the central passage 42, the fourth connecting line 59 and the branch of the first connecting line 51 connecting with the third passage outlet 2B1′ are blocked by the wall of the hollow valve core 41. At this moment, under the action of the blower 2G, the gas flow flows along a direction indicated by an arrow from the free end 53 of the first connecting line 51 to the free end 63 of the central passage 42 through the first connecting line 51, the fourth passage 41D, the fifth connecting line 61, the third passage 41C, the second connecting line 55, the blower 2G, the third connecting line 57 and the central passage 42.

When the cylindrical and hollow valve core 41 further rotates through 90° from the position as shown in FIG. 7 to a position as shown in FIG. 8, the third connecting line 57 communicates with the fourth connecting line 59 via the first passage 41A between two first holes 2A2 and 2A3, the fourth connecting line 59 communicates with the first connecting line 51 via the second passage 41B between two diametrically opposite second holes 2B2 and 2B3, the second connecting line 55 communicates with the free end 63 of the central passage 42 via the central passage, the fifth connecting line 61 and the branch of the first connecting line 51 connecting with seventh passage outlet 2D1′ are blocked by the wall of the hollow valve core 41. At this moment, under the action of the blower 2G, the gas flow flows along a direction indicated by an arrow from the free end 63 of the central passage 42 to the free end 53 of the first connecting line 51 through the central passage 42, the second connecting line 55, the blower 2G, the third connecting line 57, the first passage 41A, the fourth connecting line 59, the second passage 41B and the first connecting line 51. The gas flow is reversed at the free end 53 of the first connecting line 51 compared with that shown in FIG. 7.

When the cylindrical and hollow valve core 41 further rotates through 90° from the position as shown in FIG. 8, the reversing valve 40 returns to the position as shown in FIG. 7. This process is repeated periodically. The free end 53 of the first connecting line 51 usually couples to an airway of a patient so that the high frequency oscillation airflow generator 200 generates alternately a negative pressure and a positive pressure to deliver the gas flow from and to the patient's respiratory system, respectively. Obviously, every time the driving assembly 47 drives the cylindrical and hollow valve core 41 to rotate through 360°, the reversing valve 40 reverses the gas flow at the free end 53 of the first connecting line 51 four times. In other words, every time that the driving assembly 47 drives the cylindrical and hollow valve core 41 to rotate through 90°, the reversing valve 40 reverses the gas flow once at the free end 53 of the first connecting line 51.

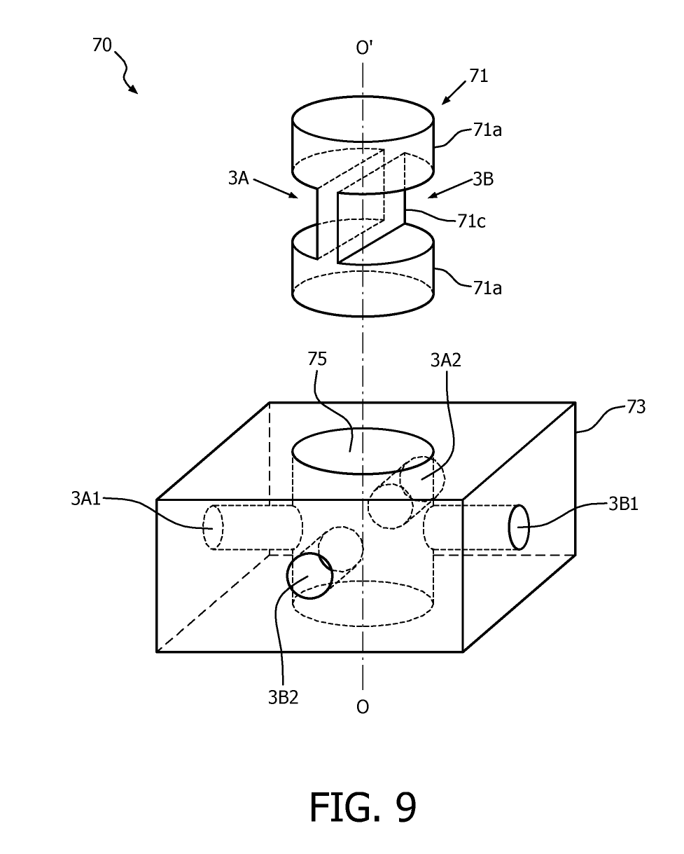

FIG. 9 is an exploded perspective view of a reversing valve according to a third embodiment of the present invention. As shown in FIG. 9, a reversing valve 70 according to a third embodiment of the present invention comprises a valve core 71. The valve core 71 comprises two circular bodies 71a and a spacer 71c centrally interconnecting said two circular bodies 71a. The two circular bodies 71a and the spacer 71c may be formed separately and then assembled together. Of course, the two circular bodies 71a and the spacer 71c may be formed integrally.

The reversing valve 70 further comprises a valve housing 73. The valve housing 73 defines a cylindrical chamber 75. Four passage outlets 3A1, 3A2, 3B1 and 3B2 opening into the cylindrical chamber 75 are formed in the valve housing 73 and distributed equidistantly along a circumferential direction. When the valve core 71 is received rotatably and hermetically within the cylindrical chamber 75 of the valve housing 73 to form the reversing valve 70, two circular bodies 71a, the spacer 71c and the valve housing 73 together define a first passage 3A and a second passage 3B.

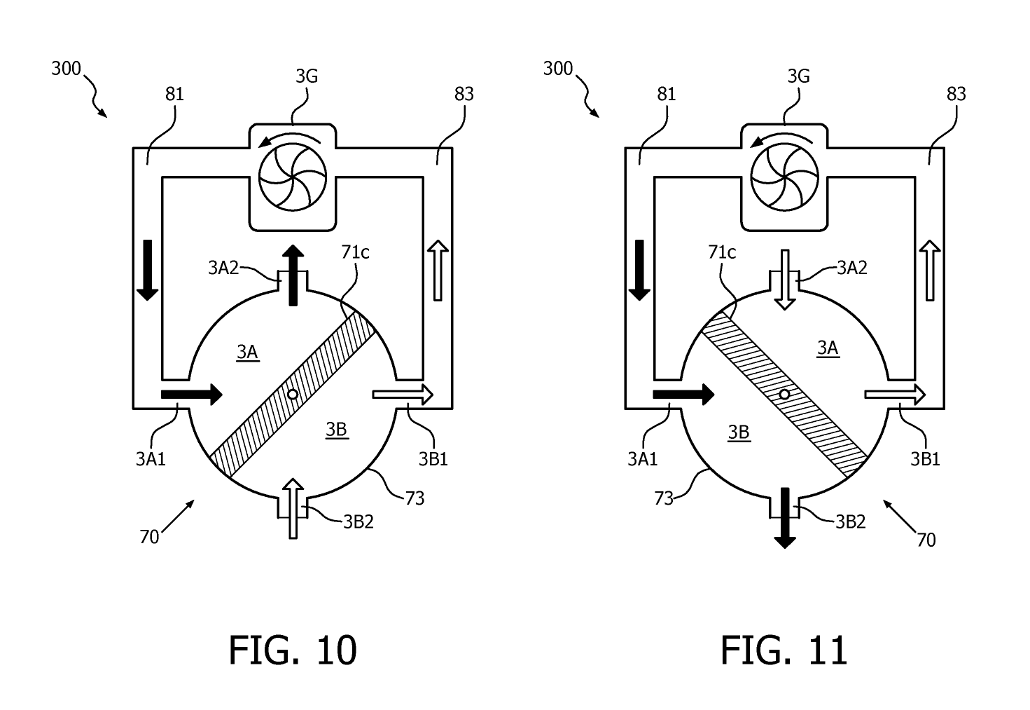

FIG. 10 is a schematic diagram of a high frequency oscillation airflow generator comprising a reversing valve according to a third embodiment of the present invention, in which the high frequency oscillation airflow generator generates a negative pressure to deliver a gas flow from a patient's respiratory system. FIG. 11 is a schematic diagram of a high frequency oscillation airflow generator comprising a reversing valve according to a third embodiment of the present invention, in which the high frequency oscillation airflow generator generates a positive pressure to deliver a gas flow to a patient's respiratory system. As shown in FIGS. 10 and 11, the high frequency oscillation airflow generator 300 comprises the reversing valve 70 according to the third embodiment of the present invention, a blower 3G, and a driving assembly (not shown in the drawings) for rotatably driving the reversing valve. The driving assembly is coupled with the valve core 71. The driving assembly may be an electrical motor. However, the driving assembly may also be a pneumatic or hydraulic motor.

The high frequency oscillation airflow generator 300 also comprises a first connecting line 81 which connects at one end with an outlet of the blower 3G and at the opposite end with the first passage outlet 3A1 of the reversing valve 70 and a second connecting line 83 which connects at one end with the second passage outlet 3B1 of the reversing valve 70 which is diametrically opposite to the first passage outlet 3A1 and at the opposite end with an inlet of the blower 3G. The third passage outlet 3A2 may open into the atmosphere while the fourth passage outlet 3B2 may open into an airway of a patient or vice versa. Of course, the third passage outlet 3A2 may open into any other suitable gas source.

When the high frequency oscillation airflow generator 300 is in operation, the driving assembly drives the valve core 71 to rotate hermetically within the valve housing 73. When the valve core 71 rotates to a position as shown in FIG. 10, under the action of the blower 3G, the gas flow flows along a direction indicated by an arrow from the fourth passage outlet 3B2 of the valve housing 73 to the third passage outlet 3A2 of the valve housing 73 through the second passage 3B, the second connecting line 83, the blower 3G, the first connecting line 81 and the first passage 3A.

When the valve core 71 further rotates through 90° from the position as shown in FIG. 10 to a position as shown in FIG. 11, the spacer 71c moves to a position perpendicular to that shown in FIG. 10. At this moment, under the action of the blower 3G, the gas flow flows along a direction indicated by an arrow from the third passage outlet 3A2 of the valve housing 73 to the fourth passage outlet 3B2 of the valve housing 73 through the first passage 3A, the second connecting line 83, the blower 3G, the first connecting line 81 and the second passage 3B.

When the valve core 71 further rotates through 90° from the position as shown in FIG. 11, the reversing valve 70 returns to the position as shown in FIG. 10. This process is repeated periodically. If the fourth passage outlet 3B2 couples to an airway of a patient, the high frequency oscillation airflow generator 300 generates alternately a negative pressure and a positive pressure to deliver the gas flow from and to the patient's respiratory system, respectively. Obviously, every time that the driving assembly drives the valve core 71 to rotate through 360°, the reversing valve 70 reverses the gas flow at the fourth passage outlet 3B2 four times. In other words, every time that the driving assembly drives the valve core 71 to rotate through 90°, the reversing valve 70 reverses the gas flow once at the fourth passage outlet 3B2.

Only one blower is required for the high frequency oscillation airflow generator according to the second and third embodiments, while two blowers are required for the high frequency oscillation airflow generator according to the first embodiment. The high frequency oscillation airflow generator according to the second and third embodiments is compact and low-weight in comparison with the high frequency oscillation airflow generator according to the first embodiment.

According to the present invention, the reversing valve reverses the gas flow generated by the high frequency oscillation airflow generator four times every time the valve core rotates through 360°. The reversing frequency of the gas flow and thus the efficiency of the high frequency oscillation airflow generator according to the present invention are twice that of the conventional pressure oscillation techniques.

The reversing frequency of the gas flow generated by the high frequency oscillation airflow generator according to the present invention may be adjusted by changing the rotating frequency of the driving assembly. The flow rate of the gas flow may be adjusted by changing the rotation speed of the blower.

In the above mentioned embodiments, the reversing valve according to the present invention is used to reverse the gas flow. It should be understood that the reversing valve according to the present invention is used to reverse a liquid flow.

Although the invention has been described in detail for the purpose of illustration, based on what is currently considered to be the most practical and preferred embodiments, it is to be understood that such detail is solely for that purpose and that the invention is not limited to the disclosed embodiments, but on the contrary, is intended to cover modifications and equivalent arrangements that are within the spirit and scope of the appended claims.

1. A reversing valve for reversing a fluid flow comprising:
a valve housing defining a cylindrical chamber, at least four passage outlets being formed in the valve housing and opening into the cylindrical chamber; and
a valve core being received rotatably and hermetically within the cylindrical chamber of the valve housing, at least two passages being defined in the valve core;
wherein the passage outlets and the passages are arranged such that the reversing valve can reverse the fluid flow once every time that the valve core rotates through 90°.

2. A reversing valve for reversing a fluid flow according to claim 1, wherein four passage outlets opening into the cylindrical chamber are formed in the valve housing, a first passage outlet and a second passage outlet thereof are diametrically opposite to each other, a third passage outlet and a fourth passage outlet thereof are diametrically opposite to each other, a line connecting the third passage outlet and the fourth passage outlet is parallel to a line connecting the first passage outlet and the second outlet; and
wherein the valve core is a cylindrical valve core, two passages are defined in the cylindrical valve core, a first passage and a second passage of the two passages are spaced from, and perpendicular to each other, both the first passage and the second passage extend through and are perpendicular to a central axis of the cylindrical valve core, and the distance between the line connecting the third passage outlet and the fourth passage outlet and the line connecting the first passage outlet and the second outlet is equal to that between the first passage and the second passage.

3. A reversing valve for reversing a fluid flow according to claim 1, wherein eight passage outlets opening into the cylindrical chamber are formed in the valve housing, a first passage outlet and a second passage outlet thereof are diametrically opposite to each other, a third passage outlet and a fourth passage outlet thereof are diametrically opposite to each other and a second line connecting the third passage outlet and the fourth passage outlet is parallel to a first line connecting the first passage outlet and the second passage outlet, a fifth passage outlet and a sixth passage outlet are diametrically opposite to each other and a third line connecting the fifth passage outlet and the sixth passage outlet is parallel to the first line, a seventh passage outlet and an eighth passage outlet are diametrically opposite to each other and a fourth line connecting the seventh passage outlet and the eighth passage outlet is parallel to the first line; and
wherein the valve core is a cylindrical and hollow valve core forming a central passage, one end of the central passage is closed and the opposite end is open, a first group of holes including four first holes, a second group of holes including two second holes, a third group of holes including four third holes, and a fourth group of holes including two fourth holes are formed in a wall of the cylindrical and hollow valve core, each group of holes is spaced from every other group of holes along a central axis of the cylindrical and hollow valve core, the four first holes are distributed equidistantly in a first plane perpendicular to the central axis of the cylindrical and hollow valve core, two diametrically opposite first holes thereof are in communication with each other via a conduit to form a first passage, the two second holes are distributed so as to be diametrically opposite in a second plane perpendicular to the central axis of the cylindrical and hollow valve core, a line connecting with the two second holes is parallel to the first passage, the two second holes are in communication with each other via a conduit to form a second passage, the four third holes are distributed equidistantly in a third plane perpendicular to the central axis of the cylindrical and hollow valve core, a line connecting with two diametrically opposite third holes thereof is perpendicular to the first passage, the two diametrically opposite third holes are in communication with each other via a conduit to form a third passage, the two fourth holes are distributed so as to be diametrically opposite in a fourth plane perpendicular to the central axis of the cylindrical and hollow valve core, a line connecting with the two fourth holes is perpendicular to the first passage, the two fourth holes are in communication with each other via as conduit to form a fourth passage, the distances between the first line and the second line, between the second line and the third line and between the third line and the fourth line are equal to those between the first plane and the second plane, between the second plane and the third plane and between the third plane and the fourth plane, respectively.

4. A reversing valve for reversing a fluid flow according to claim 1, wherein four passage outlets opening into the cylindrical chamber are formed in the valve housing and distributed equidistantly along a circumferential direction; and
wherein the valve core comprises two circular bodies and a spacer centrally interconnecting said two circular bodies when the valve core is received rotatably and hermetically within the cylindrical chamber of the valve housing to form the reversing valve, and the two circular bodies, the spacer and the valve housing together define a first passage and a second passage.

5. A high frequency oscillation airflow generator comprising a reversing valve for reversing a gas flow, at least one blower, a driving assembly for rotatably driving the reversing valve and a plurality of connecting lines connecting the reversing valve with the at least one blower, the reversing valve comprising:
a valve housing defining a cylindrical chamber, at least four passage outlets being formed in the valve housing and opening into the cylindrical chamber; and
a valve core being received rotatably and hermetically within the cylindrical chamber of the valve housing, at least two passages being defined in the valve core;
wherein the passage outlets and the passages are arranged such that the reversing valve can reverse the gas flow once every time that the driving assembly drives the valve core to rotate through 90°.

6. A high frequency oscillation airflow generator according to claim 5, wherein four passage outlets opening into the cylindrical chamber are formed in the valve housing, a first passage outlet and a second passage outlet thereof are diametrically opposite to each other, a third passage outlet and a fourth passage outlet thereof are diametrically opposite to each other, a line connecting the third passage outlet and the fourth passage outlet is parallel to a line connecting the first passage outlet and the second outlet;
wherein the valve core is a cylindrical valve core, two passages are defined in the cylindrical valve core, a first passage and a second passage of the two passages are spaced from and perpendicular to each other, both the first passage and the second passage extend through and are perpendicular to a central axis of the cylindrical valve core, and the distance between the line connecting the third passage outlet and the fourth passage outlet and the line connecting the first passage outlet and the second outlet is equal to that between the first passage and the second passage; and
wherein the at least one blower comprises a first blower and a second blower, the plurality of connecting lines comprises a first connecting line, a second connecting line and a third connecting line, the first connecting line has a free end and connects at the opposite end with the second passage outlet and the fourth outlet, the second connecting line connects at one end with the first passage outlet and at the opposite end with an outlet of the first blower, the third connecting line connects at one end with the third passage outlet and at the opposite end with an inlet of the second blower an inlet of the first blower and an outlet of the second blower open into the atmosphere or other gas source.

7. A high frequency oscillation airflow generator according to claim 5, wherein eight passage outlets opening into the cylindrical chamber are formed in the valve housing, a first passage outlet and a second passage outlet thereof are diametrically opposite to each other, a third passage outlet and a fourth passage outlet thereof are diametrically opposite to each other and a second line connecting the third passage outlet and the fourth passage outlet is parallel to a first line connecting the first passage outlet and the second passage outlet, a fifth passage outlet and a sixth passage outlet are diametrically opposite to each other and a third line connecting the fifth passage outlet and the sixth passage outlet is parallel to the first line, a seventh passage outlet and an eighth passage outlet are diametrically opposite to each other and a fourth line connecting the seventh passage outlet and the eighth passage outlet is parallel to the first line;
wherein the valve core is a cylindrical and hollow valve core forming a central passage, one end of the central passage is closed and the opposite end is open, a first group of holes including four first holes, a second group of holes including two second holes, a third group of holes including four third holes, and a fourth group of holes including two fourth holes are formed in a wall of the cylindrical and hollow valve core, each group of holes is spaced from every other group of holes along a central axis of the cylindrical and hollow valve core, the four first holes are distributed equidistantly in a first plane perpendicular to the central axis of the cylindrical and hollow valve core, two diametrically opposite first holes thereof are in communication with each other via a conduit to form a first passage, the two second holes are distributed so as to be diametrically opposite in a second plane perpendicular to the central axis of the cylindrical and hollow valve core, a line connecting with the two second holes is parallel to the first passage, the two second holes are in communication with each other via a conduit to form a second passage, the four third holes are distributed equidistantly in a third plane perpendicular to the central axis of the cylindrical and hollow valve core, a line connecting with two diametrically opposite third holes thereof is perpendicular to the first passage, the two diametrically opposite third holes are in communication with each other via a conduit to form a third passage, the two fourth holes are distributed so as to be diametrically opposite in a fourth plane perpendicular to the central axis of the cylindrical and hollow valve core, a line connecting with the two fourth holes is perpendicular to the first passage, the two fourth holes are in communication with each other via a conduit to form a fourth passage, the distances between the first line and the second line, between the second line and the third line and between the third line and the fourth line are equal to those between the first plane and the second plane, between the second plane and the third plane and between the third plane and the fourth plane, respectively;
wherein the at least one blower comprises one blower, the plurality of connecting lines comprise a first connecting line, a second connecting line, a third connecting line, a fourth connecting line and a fifth connecting line, the first connecting line has a free end and connects at the opposite end with the third passage outlet and the seventh passage outlet, the second connecting line connects at one end with the fifth passage outlet and at the opposite end with an inlet of the blower, the third connecting line connects at one end with an outlet of the blower and at the opposite end with the first passage outlet, the fourth connecting line connects at one end with the second passage outlet and at the opposite end with the fourth passage outlet, and the fifth connecting line connects at one end with the sixth passage outlet and at the opposite end with the eighth passage outlet, and the free end of the central passage of the hollow valve core opens into the atmosphere or other gas source.

8. A high frequency oscillation airflow generator according to claim 5, wherein four passage outlets opening into the cylindrical chamber are formed in the valve housing and distributed equidistantly along a circumferential direction;
wherein the valve core comprises two circular bodies and a spacer centrally interconnecting said two circular bodies when the valve core is received rotatably and hermetically within the cylindrical chamber of the valve housing to form the reversing valve, the two circular bodies, the spacer and the valve housing together define a first passage and a second passage; and
wherein the at least one blower comprises one blower, the plurality of connecting lines comprises a first connecting line and a second connecting line, the first connecting line connects at one end with an outlet of the blower and at the opposite end with a first passage outlet of the four passage outlets, the second connecting line connects at one end with a second passage outlet of the four passage outlets which is diametrically opposite to the first passage outlet and at the opposite end with an inlet of the blower, and one of a third passage outlet and a fourth passage outlet remains unobstructed and the other one opens into the atmosphere or other gas source.

9. A high frequency oscillation airflow generator according to claim 5, wherein a reversing frequency of the gas flow generated by the high frequency oscillation airflow generator is adjusted by changing the rotating frequency of the driving assembly.

10. A high frequency oscillation airflow generator according to claim 5, wherein the flow rate of the gas flow generated by the high frequency oscillation airflow generator is adjusted by changing the rotation speed of the blower.
